(19) United States
(12) Patent Application Publication
(10) Pub. No.: US 2009/0147531 A1
(43) Pub. Date: Jun. 11, 2009

(54) ORAL ILLUMINATOR

(75) Inventors: Chih-Peng Hsu, Chu-Nan (TW); Tse-An Lee, Chu-Nan (TW)

Correspondence Address: PCE INDUSTRY, INC.;ATT. Steven Reiss, 458 E. LAMBERT ROAD, FULLERTON, CA, 92835, US

(73) Assignee: FOXSEMICON INTEGRATED TECHNOLOGY, INC., Chu-Nan (TW)

(21) Appl. No.: 12/176,252

(22) Filed: Jul. 18, 2008

(30) Foreign Application Priority Data
Dec. 6, 2007 (CN) 200710202895.7

(51) Int. Cl. A61B 1/06; A61C 19/00

(52) U.S. Cl. 362/573; 433/29

(57) ABSTRACT

An oral illuminator includes a solid state light-emitting element emitting light, an optical fiber, and a light diffuser. The optical fiber is arranged between the solid state light-generating element and the diffuser for transmitting the light of the solid state light-generating element to the diffuser. The optical fiber has an incident surface optically coupled to the solid state light-generating element, and an emitting surface optically coupled to the diffuser.

### BACKGROUND

1. Field of the Invention

The present invention relates to an oral illuminator, and particularly to an oral illuminator incorporating a solid state lighting element as a light source.

2. Description of Related Art

Generally, light sources used during dental procedures rely upon incandescent light sources, such as halogen-based light bulbs, or arc lamps, such as xenon or mercury arc lamps. However, both incandescent and xenon sources consume a great amount of power, typically 250 W or more, and thus generate extreme heat. Other light sources for dental procedures are solid state devices, such as light emitting diodes (LEDs). Each of the LEDs is preferably coupled to one end of a single optical fiber. The optical cable transmits the light of the LEDs to a dental lighting handpiece. However, the light emitted from the optical fiber is usually unidirectional, and thus only part of the oral cavity is illuminated, which is inconvenient for the dental procedures.

For the foregoing reasons, there is a need in the art for an oral illuminator which overcomes the limitations described.

### SUMMARY

According to an exemplary embodiment of the present invention, an oral illuminator includes a solid state light-generating element emitting light, an optical fiber, and a light diffuser. The optical fiber is arranged between the solid state light-generating element and the diffuser transmits light therebetween. The optical fiber has an incident surface optically coupled to the solid state light-generating element, and an emitting surface optically coupled to the light diffuser.

Other advantages and novel features of the present invention will be drawn from the following detailed description of the exemplary embodiments of the present invention with attached drawings.

### BRIEF DESCRIPTION OF THE DRAWINGS

FIG. 1 is a schematic view of an oral illuminator according to an exemplary embodiment of the present invention.

FIG. 2 is a graph indicating an optical pathway of an optical fiber of the oral illuminator of FIG. 1.

FIGS. 3-4 show a diffuser of the oral illuminator in different shapes.

FIG. 5 shows an alternative embodiment of the oral illuminator.

FIG. 6 shows a third embodiment of the oral illuminator.

FIG. 7 shows the oral illuminator according to a fourth embodiment.

FIG. 8 shows a fifth embodiment of the oral illuminator.

### DETAILED DESCRIPTION OF THE EMBODIMENTS

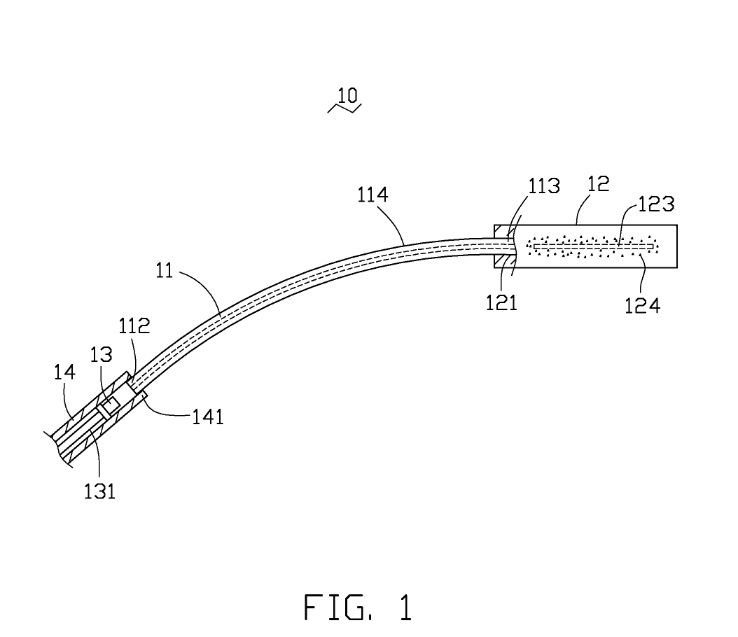

Referring to FIG. 1, an oral illuminator 10 according to an exemplary embodiment includes a solid state light generating element emitting light, an optical fiber 11 and a diffuser 12.

In this embodiment, a light emitting diode (LED) 13 is adopted as the solid state light-generating element for its small size, long lifespan and high efficiency. Alternatively, a plurality of LEDs 13 may be used as a light source to emit sufficient light when a high intensity of light is needed. A pair of threads 131 extends from the LED 13, electrically connecting the LED 13 to a power source (not shown). Thus, electrical current is supplied to the LED 13 to generate light. A shell 14 surrounds the LED 13 protecting the LED 13 from damage and contamination. The shell 14 has an open end 141 at one side thereof. The LED 13 has a light emitting surface (not labeled) facing the open end 141.

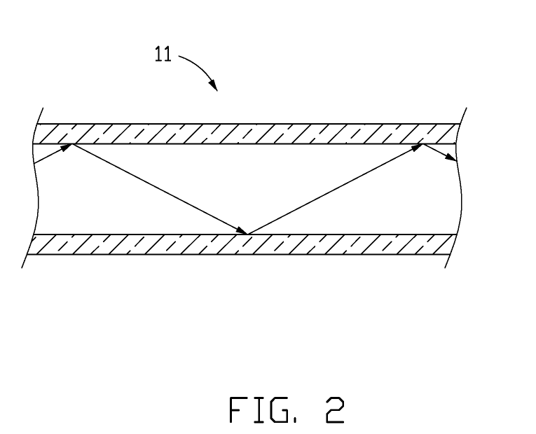

The optical fiber 11 is transparent glass or plastic, and is configured to transmit light from the LED 13. According to the principle of total internal reflection, when an angle of incidence of the light exceeds a critical value, the light will be completely reflected, and thus cannot escape. FIG. 2 shows an optical pathway of the light of the LED 13 traveling through the optical fiber 11. Total internal refection confines the light within the optical fiber 11. The light travels along the optical fiber 11 bouncing off the boundaries of the optical fiber 11, and the light is guided from one end to the other end with minimal loss. In this embodiment, the optical fiber 11 is arranged between and optically couples to the LED 13 and the diffuser 12. One end of the optical fiber 11 is received in the open end 141 of the shell 14. An outer surface of the end of the optical fiber 11 optically couples to the emitting surface of the LED 13 and thus forms an incident surface 112 of the optical fiber 11. Another end of the optical fiber 11 is received in the diffuser 12. An outer surface of the opposite end of the optical fiber 11 optically couples to the diffuser 12, forming an emitting surface 112 of the optical fiber 11. An elongated tube 114 of soft plastic is configured for receiving the optical fiber 11, providing protection from being damaged. A flexible metal strip (not shown) can be arranged in the tube 114 to improve flexibility of the optical fiber 11.

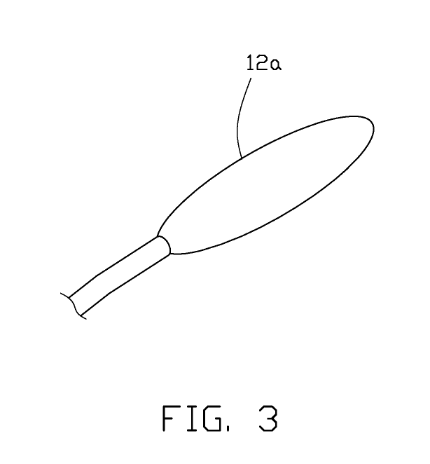

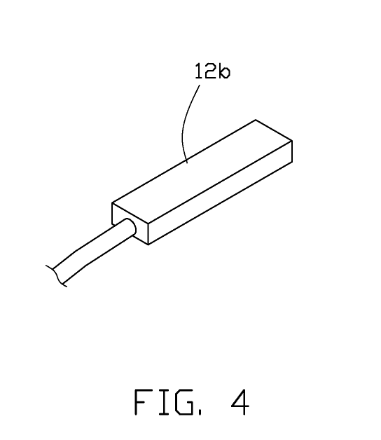

The diffuser 12 is transparent material, such as soft plastic, rigid plastic, silica gel, glass, quartz, acrylic or other. In this embodiment, the diffuser 12 is soft plastic, and a flexible metal strip 123 is arranged in the diffuser 12 to improve flexibility thereof. The diffuser 12 is columnar, but, alternatively, can be other shapes. As shown in FIG. 3, the diffuser 12a can be ovoid, or, as in FIG. 4, the diffuser 12b can be planar. A plurality of granules 124 are dispersed in the diffuser 12 for enhancing scattering effect of the diffuser 12. The granules 124 can be crystalline particles or metal particles. Alternatively, the granules 124 can be replaced by a plurality of air bladders. In addition, a plurality of V-cut or scattering dots can be formed on the outer surface of the diffuser 12 for enhancing scattering effect. A mounting hole 121 is defined in the diffuser 12 for engagement of the optical fiber 11. Thus, the diffuser 12 can be attached to or detached from the oral illuminator 10 conveniently, easing replacement thereof.

When the oral illuminator 10 is in use, the diffuser 12 is inserted into a patient's oral cavity. The LED 13 is electrically connected to the power source, electrical current is supplied by the power source to light the LED 13. The light of the LED 13 enters the optical fiber 11 through the incident surface 112 thereof, and then travels along the optical fiber 11 to the emitting surface 112 thereof with minimal loss. Finally the light enters the diffuser 12. The granules 124 arranged in the diffuser 12 and the V-cut or scattering dots formed on the outer surface of the diffuser 12 cause the light to diffuse and emit from the outer surface of the diffuser 12 in all directions, illuminating much of the oral cavity. As the diffuser 12 is made of soft plastic, the diffuser 12 can be take different shapes according to needs.

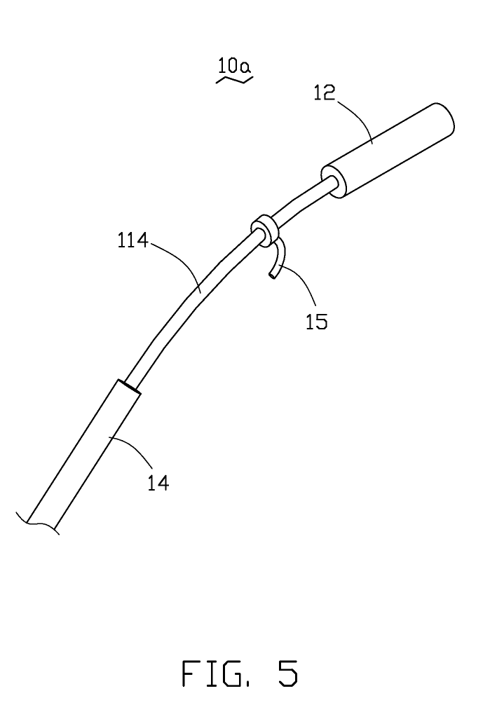

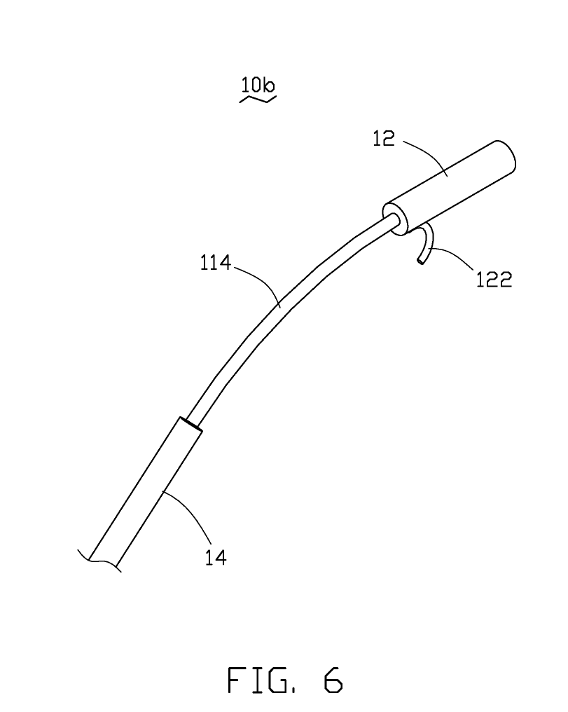

FIGS. 5-8 show alternative embodiments of the oral illuminators 10a, 10b, 10c, 20. As shown in FIG. 5, a hook 15 is formed on the tube 114, configured for hanging the oral illuminator 10a, such as on a mouth of a dental patient. The hook 15 is moveable along the tube 114, thus the position of the diffuser 12 can be adjusted by moving the hook 15. FIG. 6 shows the hook 122 in a different position, here on the diffuser 12 near the emitting surface 112 of the optical fiber 11. Thus, after the oral illuminator 10b is used, the hook 122 formed on the diffuser 12 can be replaced with the diffuser 12.

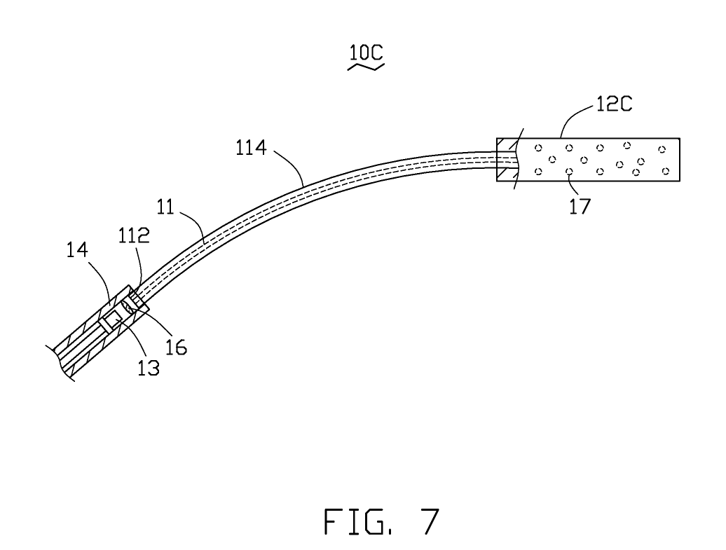

FIG. 7 shows a third embodiment of the oral illuminator 10c, differing from the first embodiment in that a lens 16 is arranged between the incident surface 112 of the optical fiber 11 and the emitting surface of the LED 13c for converging the light of the LED 13c. As a result, almost all of the light emitted from the LED 13c can enter the optical fiber 11 and be transmitted to the diffuser 12 to illuminate the oral cavity. The LED 13c can be blue, purple, or ultraviolet, or others. A plurality of yellow phosphor particles 17 are arranged in the diffuser 12c. After the light of the LED 13c enters the diffuser 12c, a part of the light of the LED 13c absorbed by the phosphor particles and converted to yellow light. The remaining part of the light of the LED 13c mixes with the yellow light and is perceived as white light.

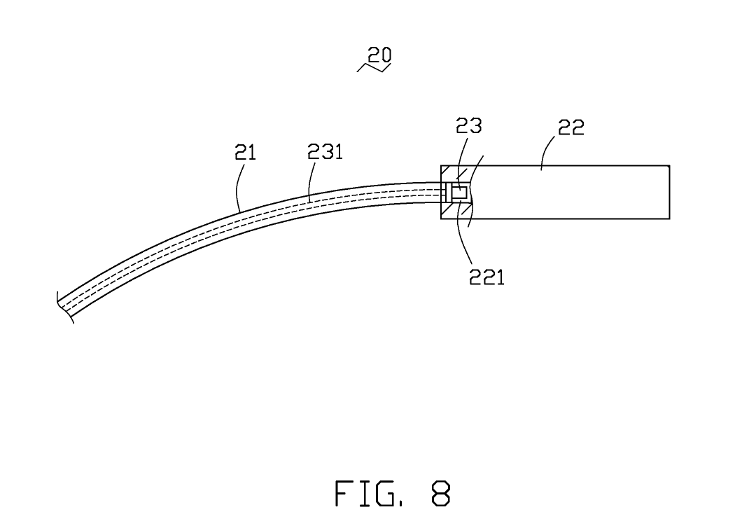

FIG. 8 shows an oral illuminator 20 without optical fiber 11. Similarly, the LED 23 is received in a shell 21, and has a pair of threads 231 extending outwardly for electrically connecting the LED 23 with the power source. The difference is that the LED 23 with the surrounding shell 21 is received in a mounting hole 221 of the diffuser 22 to directly emit light into the diffuser 22. Thus, loss of light during transmission is avoided.

It is understood that the invention may be embodied in other forms without departing from the spirit thereof. Thus, the present examples and embodiments are to be considered in all respects as illustrative and not restrictive, and the invention is not to be limited to the details given herein.

What is claimed is:

1. An oral illuminator, comprising:
a solid state light-generating element emitting light;
a diffuser for diffusing the light of the solid state light-generating element; and
an optical fiber between the solid state light-generating element and the diffuser for transmitting the light of the solid state light-generating element to the diffuser, the optical fiber having an incident surface optically coupled to the solid state light-generating element, and an emitting surface optically coupled to the diffuser.

2. The oral illuminator of claim 1, further comprising a tube receiving the optical fiber therein.

3. The oral illuminator of claim 2, further comprising a hook formed on the tube and being movable relative to the tube.

4. The oral illuminator of claim 1, wherein a hook is formed on the diffuser near the emitting surface of the optical fiber.

5. The oral illuminator of claim 1, further comprising a lens between the incident surface of the optical fiber and the solid state light-generating element.

6. The oral illuminator of claim 1, wherein the diffuser is columnar, ovoid, or planar.

7. The oral illuminator of claim 1, wherein the diffuser has a plurality of crystalline particles or metal particles arranged therein.

8. The oral illuminator of claim 1, wherein a plurality of V-cut or scattering dots are formed on an outer surface of the diffuser.

9. The oral illuminator of claim 1, further comprising a flexible metal strip arranged in the diffuser.

10. The oral illuminator of claim 1, wherein the diffuser is detachable.

11. The oral illuminator of claim 1, wherein the diffuser has a plurality of phosphor particles arranged therein.

12. The oral illuminator of claim 1, wherein the solid state light-generating element comprises at least one light emitting diode.

13. An oral illuminator, comprising:
a solid state light-generating element emitting light;
a shell surrounding the solid state light-generating element; and
a diffuser optically coupled to the solid state light-generating element for diffusing the light of the solid state light-generating element, the light emitting from the diffuser in all directions.

14. The oral illuminator of claim 13, wherein the solid state generating element comprises at least one light emitting diode.

15. The oral illuminator of claim 13, wherein a mounting hole is defined in the diffuser, receiving the solid state light-generating element.

16. The oral illuminator of claim 13, wherein an optical fiber interconnects the solid state light-generating element and the diffuser, the diffuser defining a mounting hole receiving an emitting end of the optical fiber, an incidence end of the optical fiber being received in the shell and facing an emitting surface of the solid state light-generating element.

17. The oral illuminator of claim 13, wherein the diffuser is columnar, ovoid, or planar.

18. The oral illuminator of claim 13, wherein the diffuser has a plurality of crystalline particles or metal particles arranged therein, and a plurality of V-cut or scattering dots are formed on an outer surface of the diffuser.

19. The oral illuminator of claim 13, wherein a hook is formed on an end of the diffuser near the solid state light-generating element.
